(19) United States
(12) Patent Application Publication
(10) Pub. No.: US 2023/0072138 A1
(43) Pub. Date: Mar. 9, 2023

(54) SURGICAL FASTENER HAVING A BASE AND A LEG

(71) Applicant: Covidien LP, Mansfield, MA (US)

(72) Inventor: Olesea Diaz-Chiosa, Naugatuck, CT (US)

(21) Appl. No.: 17/468,893

(22) Filed: Sep. 8, 2021

(51) Int. Cl. A61B 17/064

(52) U.S. Cl. CPC A61B 17/0644; A61B 2017/0645; A61B 2017/00867

(57) ABSTRACT

A surgical fastening assembly includes first and second surgical fasteners. Each surgical fastener includes a base with opposed first and second ends. The base is formed from a deformable material. Each surgical fastener also includes a leg with a proximal end resiliently coupled to the second end of its respective base and a distal tip configured to penetrate body tissue. The first ends of the first and second surgical fasteners are in an abutting relationship. The distal tip of the leg of the first surgical fastener is adjacent the second end of the second surgical fastener and the distal tip of the leg of the second surgical fastener is adjacent the second end of the first surgical fastener such that the legs cross and define an X-shape.

### FIELD

The present disclosure generally relates to a surgical fastener. In particular, the present disclosure relates to a surgical fastener having a base and a leg.

### BACKGROUND

Surgical fasteners have been used to reduce the need for suturing, which is both time consuming and inconvenient. In many applications the surgeon can use a surgical apparatus, i.e., a fastener implanting device, loaded with surgical fasteners to accomplish in a few seconds what would have taken many minutes to perform by suturing. This reduces blood loss and trauma to the patient.

During a surgical procedure, body tissue is resected and apposed to form an anastomosis. Anastomosis can be facilitated by the use of mechanical fasteners, which provide a standardized closure and hemostasis in a wide range of tissues.

Surgical fasteners have been in the form of ordinary metal staples, which are bent by the delivery apparatus to hook together body tissue. Typically, staples have a pair of legs joined together at one end by a crown. They are designed so that they may be deformed to hold body tissue. Accordingly, the staplers have embodied structure functioning to project the conventional staple into tissue as well as to deform the staple so that it is retained against the tissue. In some applications, access to the body tissue from two opposite directions is available and an anvil can operate to deform the legs of the staple after they have passed through the body tissue. In applications where access to the tissue is from only one direction, the anvil may deform the crown of the conventional staple so that its legs will project into the body tissue in a fashion so as to hold the staple in the tissue.

Two-part fasteners have also been utilized, where a barbed staple is used in conjunction with a retaining piece to hold the staple in place. Typically, the two-part fastener has a crown or backspan and two barbed prongs which are engaged and locked into a separate retainer piece. In use, the staple is pressed into the body tissue so that the barbs penetrate the tissue and emerge from the other side where they are then locked into the retainer piece. Retainers prevent the staple from working loose from the tissue. The two-piece fasteners cannot be unlocked and are not removable.

Like other applications, however, the two-piece fasteners require the staple delivery apparatus to have access to both sides of the tissue. Thus, as with the other applications, two-piece fasteners are limited since they cannot be used where access to tissue is from one direction only.

In those situations where access to body tissues is limited to one direction, as in grafting procedures, deformable surgical fasteners have been employed. As mentioned previously, however, the applicators commonly used in these situations embody an anvil cooperating with a fastener to deform it and consequently, tend to be of a complex design.

Thus, a surgical fastener that does not need access to both sides of body tissue for installation is desirable.

### SUMMARY

In accordance with an aspect of the present disclosure, a surgical fastener has a base with opposed first and second ends. A leg extends from the first end of the base and includes a distal tip configured to penetrate body tissue. The distal tip is configured to extend towards the second end of the base. A length of the leg defines an acute angle with respect to a longitudinal axis of the base.

In one aspect of the present disclosure, the leg may be resiliently coupled to the base.

In an aspect of the present disclosure, slidable engagement between the base and an actuator rod may deflect the distal tip away from the second end of the base.

In aspects of the present disclosure, the base may be formed of a deformable material such that slidable engagement between the base and the actuator rod deforms the base thereby deflecting the distal tip of the leg.

In another aspect of the present disclosure, the leg of the first surgical fastener may be configured to couple with a second surgical fastener having a base with opposed first and second ends and a leg resiliently coupled to the second end of the base.

In a further aspect of the present disclosure, the distal tip of the leg may contact the second end of the base of the second surgical fastener.

In a further aspect of the present disclosure, the surgical fastener may be formed from a shape memory material.

In accordance with another aspect of the present disclosure, a surgical fastening assembly includes a first surgical fastener having a base and a leg. The base is formed from a deformable material and has opposed first and second ends. The leg has a distal tip and a proximal end resiliently coupled to the second end of the base. A second surgical fastener has a base and a leg. The base is formed from a deformable material and has opposed first and second ends. The leg has a distal tip and a proximal end resiliently coupled to the second end of the base. Slidable engagement between the base of the first surgical fastener and an actuator rod deforms the base of the first surgical fastener thereby deflecting the distal tip of the leg of the first surgical fastener. Slidable engagement between the base of the second surgical fastener and the actuator rod deforms the base of the second surgical fastener thereby deflecting the distal tip of the leg of the second surgical fastener.

In one aspect of the present disclosure, slidable engagement between the actuator rod and the base of the second surgical fastener may allow the distal tip of the leg of the first surgical fastener to transition to a rest position such that the distal tip of the leg of the first surgical fastener is proximate the second end of the base of the second surgical fastener.

In another aspect of the present disclosure, the legs of the first and second surgical fasteners may define acute angles with respect to a longitudinal axis of the first and second surgical fasteners.

In aspects of the present disclosure, the distal tips of the legs may be configured to penetrate body tissue.

In another aspect of the present disclosure, a mid point of the legs may be orthogonal to junction between the first ends of the bases of the first and second surgical fasteners thereby defining a symmetrical surgical fastening assembly.

In a further aspect of the present disclosure, the legs may cross one another and define an X-shape.

In yet another aspect of the present disclosure, the first ends of the first and second surgical fasteners may be in an abutting relationship.

In an aspect of the present disclosure, the first and second surgical fasteners may be formed from a shape memory material.

According to an aspect of the present disclosure, a method of forming a surgical fastening assembly includes positioning a first surgical fastener proximate a second surgical fastener where each surgical fastener has a base and a leg. The method also includes the bases having opposed first and second ends with the legs resiliently coupled to the second ends of the bases. The method includes translating an actuator rod and engaging the base of the first surgical fastener thereby deforming the base of the first surgical fastener and deflecting a distal tip of the leg of the first surgical fastener away from the first end of the base of the first surgical fastener. Additionally, the method includes translating the actuator rod and engaging the base of the second surgical fastener thereby deforming the base of the second surgical fastener and deflecting a distal tip of the leg of the second surgical fastener away from the first end of the base of the second surgical fastener and allowing the distal tip of the leg of the first surgical fastener to transition towards the second end of the base of the second surgical fastener. Further, the method includes retracting the actuator rod thereby allowing the distal tip of the leg of the second surgical fastener to transition towards the second end of the first surgical fastener.

In an aspect of the present disclosure, positioning the first surgical fastener proximate the second surgical fastener may include distal tips of the legs being configured to penetrate tissue.

In aspects of the present disclosure, translating the actuator rod and engaging the base of the second surgical fastener may urge the first ends of the first and second surgical fasteners into an abutting relationship.

In another aspect of the present disclosure, retracting the actuator rod may position a mid point of the legs orthogonal to a junction between the first ends of the bases of the first and second surgical fasteners thereby defining a symmetrical surgical fastening assembly.

In a further aspect of the present disclosure, retracting the actuator rod may position the legs of the first and second surgical fasteners across one another thereby defining an X-shape.

In yet another aspect of the present disclosure, retracting the actuator rod may position the distal tips of the legs adjacent the second ends of the bases of the first and second surgical fasteners.

In aspects of the present disclosure, retracting the actuator rod may position the distal tips of the legs in contact with the second ends of the bases of the first and second surgical fasteners.

According to an aspect of the present disclosure, a surgical system for deploying a surgical fastener includes an end effector having first and second jaws, a ribbon supporting a surgical fastener thereon, the surgical fastener having a central portion with opposed first and second legs, and first and second rollers disposed in the first and second jaws respectively, the first and second rollers attached to respective first and second cables, the first roller configured to engage the first leg of the surgical fastener and the second roller configured to engage the second leg of the surgical fastener such that proximal movement of the first and second rollers transitions the surgical fastener from an undeployed configuration to a deployed configuration.

In aspects of the present disclosure, the surgical fastener may be capable of joining adjacent layers of tissue in the deployed configuration.

In another aspect of the present disclosure, the surgical fastener may have an S-shaped configuration in the deployed configuration.

In a further aspect of the present disclosure, the surgical fastener may be formed from a shape memory material.

In another aspect of the present disclosure, transitioning the surgical fastener from the undeployed configuration to the deployed configuration may separate the surgical fastener from the ribbon.

Other features of the disclosure will be appreciated from the following description.

### DESCRIPTION OF THE DRAWINGS

The accompanying drawings, which are incorporated in and constitute a part of this specification, illustrate aspects and features of the disclosure and, together with the detailed description below, serve to further explain the disclosure, in which:

FIG. 1 is a side view of a distal portion of an actuator and first and second surgical fasteners according to an aspect of the present disclosure;

FIG. 2 is a side view of the distal portion of the actuator engaging the first surgical fastener;

FIG. 3 is a side view of the first and second surgical fasteners joining layers of body tissue;

FIG. 4 is a top view of legs of the first and second surgical fasteners;

FIG. 5 is a bottom view of bases of the first and second surgical fasteners;

FIG. 6A is a side view of an actuator and a surgical fastener prior to deployment into body tissue according to another aspect of the present disclosure;

FIG. 6B is a side view of the actuator and the surgical fastener of FIG. 6A showing an initial phase of deployment;

FIG. 6C is a side view of the actuator and the surgical fastener of FIG. 6B showing an intermediate phase of deployment;

FIG. 6D is a side view of the actuator and the surgical fastener of FIG. 6C showing a final phase of deployment;

FIG. 7 is a side partial cut away view of an end effector engaging layers of body tissue and a ribbon supporting surgical fasteners thereon according to a further aspect of the present disclosure;

FIG. 8 is a side partial cut away view of the end effector of FIG. 7 illustrating deployment of the surgical fasteners; and

FIG. 9 is a side cut away view of the surgical fasteners joining layers of body tissue.

### DETAILED DESCRIPTION

Aspects of the disclosure are described hereinbelow with reference to the accompanying drawings; however, it is to be understood that the disclosed aspects are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure.

Descriptions of technical features of an aspect of the disclosure should typically be considered as available and applicable to other similar features of another aspect of the disclosure. Accordingly, technical features described herein according to one aspect of the disclosure may be applicable to other aspects of the disclosure, and thus duplicative descriptions may be omitted herein. Like reference numerals may refer to like elements throughout the specification and drawings.

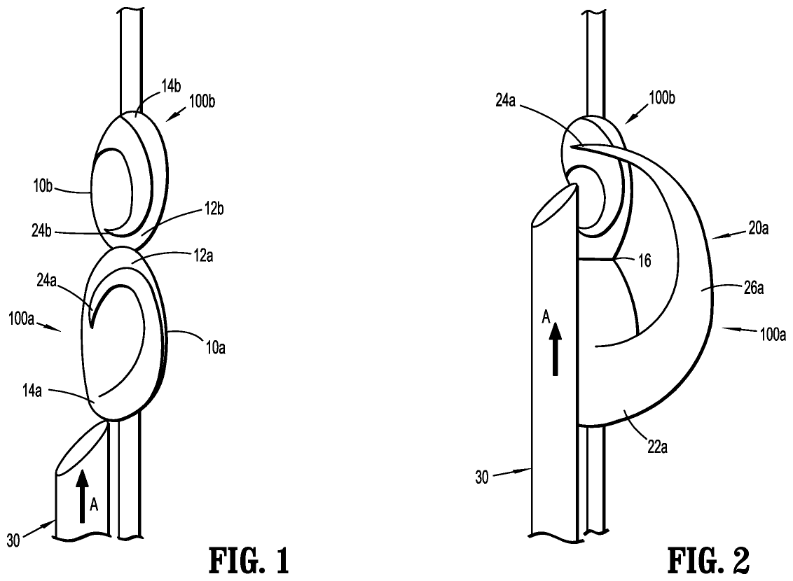

Initially, with reference to FIGS. 1-3, the presently disclosed surgical fastener is illustrated and identified as surgical fastener 100. In particular, two surgical fasteners 100a, 100b are shown adjacent to one another in proximity to a distal end of an actuator rod 30. The two surgical fasteners 100a, 100b are employed to form a surgical fastening assembly 10 as will be explained hereinbelow. The surgical fasteners 100a, 100b include bases 10a, 10b having first ends 12a, 12b and opposed second ends 14a, 14b that define a longitudinal axis L-L of the bases 10a, 10b. The bases 10a, 10b are formed from a suitable biocompatible material and are at least partially deformable. The surgical fasteners 100a, 100b may be formed from a material with shape memory properties such as a shape memory metal (e.g., a nickel-titanium alloy or a copper-aluminum-nickel alloy) or a shape memory polymer. The surgical fasteners 100a, 100b also include legs 20a, 20b having proximal ends 22a, 22b and distal tips 24a, 24b at opposed ends of arcuate arms 26a, 26b. The arcuate arms 26a, 26b define a length of the legs 20a, 20b. The legs 20a, 20b have an arcuate configuration with the proximal ends 22a, 22b of the legs 20a, 20b being resiliently attached to the second ends 14a, 14b of the bases 10a, 10b. In an initial or rest position, the legs 20a, 20b extend along a majority of a length of the bases 10a, 10b such that the distal tips 24a, 24b of the legs 20a, 20b are located near the first ends 12a, 12b of their respective bases 10a, 10b. The distal tips 24a, 24b of the legs 20a, 20b have points and are configured to penetrate body tissue. Each leg 20a, 20b defines an axis X-X.

The surgical fasteners 100a, 100b are transitionable between the rest position (FIG. 1) and an extended position (FIG. 2). As shown in FIGS. 1 and 2, the surgical fasteners 100a, 100b are positioned adjacent one another with their respective first ends 12a, 12b in an abutting relationship. In this configuration, the distal tips 24a, 24b of the legs 20a, 20b are proximate one another. As the actuator rod 30 is translated in the direction of arrow “A”, the actuator rod 30 slidably engages the second end 14a of the base 10a of the surgical fastener 100a causing the base 10a of the surgical fastener 100a to deform. As the base 10a of surgical fastener 100a deforms, the leg 20a of surgical fastener 100a deflects and transitions from the rest position towards the extended position. As the actuator rod 30 continues translation in the direction of arrow “A”, the resiliency of the leg 20a urges the leg 20a back towards the rest position. The continued translation of the actuator rod 30 in the direction of arrow “A” slidably engages the first end 12b of the surgical fastener 100b. As the actuator rod 30 engages the first end 12b of the surgical fastener 100b, the base 10a of surgical fastener 100a remains deformed such that the distal tip 24a of the leg 20a of surgical fastener 100a is now proximate the second end 14b of the base 10b of surgical fastener 100b. In a similar fashion to the interaction between the actuator rod 30 and the surgical fastener 100a, the slidable engagement between the actuator rod 30 and the base 10b of the surgical fastener 100b deforms the base 10b and the leg 20b of the surgical fastener 100b deflects and transitions from the rest position towards the extended position. As the actuator rod 30 continues movement relative to the surgical fastener 100b, the base 10b of surgical fastener 100b remains deformed such that the distal tip 24b of the leg 20b of surgical fastener 100b is proximate the second end 14a of the surgical fastener 100a.

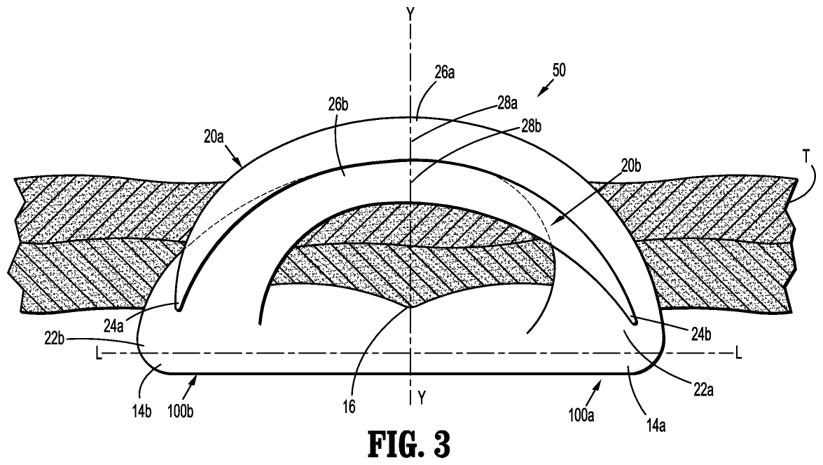

With reference now to FIGS. 3-5, the surgical fasteners 100a, 100b are shown coupled together to form the surgical fastening assembly 50. The second ends 14a, 14b of the surgical fasteners 100a, 100b are at opposed ends of the surgical fastening assembly 50 while the first ends 12a, 12b of the surgical fasteners 100a, 100b are in an abutting relationship. In particular, the first ends 12a, 12b of the surgical fasteners 100a, 100b are deformed as a result of the interaction between the actuator rod 30 and the bases 10a, 10b of the surgical fasteners 100a, 100b (see FIG. 5). During the transition from the rest position to the extended position, the legs 20a, 20b of the surgical fasteners 100a, 100b penetrate through adjacent layers of body tissue T and subsequently transition towards the rest position. As the first ends 12a, 12b of the bases 10a, 10b of the surgical fasteners 100a, 100b are deformed by their interaction with the actuator rod 30, the overall length of the surgical fastening assembly 50 is less than the sum of the lengths of the bases 10a, 10b of the surgical fasteners 100a, 100b. Further, the distal tips 24a, 24b of the legs 20a, 20b of the surgical fasteners 100a, 100b are in contact with the second ends 14a, 14b of the surgical fasteners 100a, 100b. In particular, the distal tip 24a of the leg 20a of the surgical fastener 100a contacts the second end 14b of the base 10b of the surgical fastener 100b and vice versa.

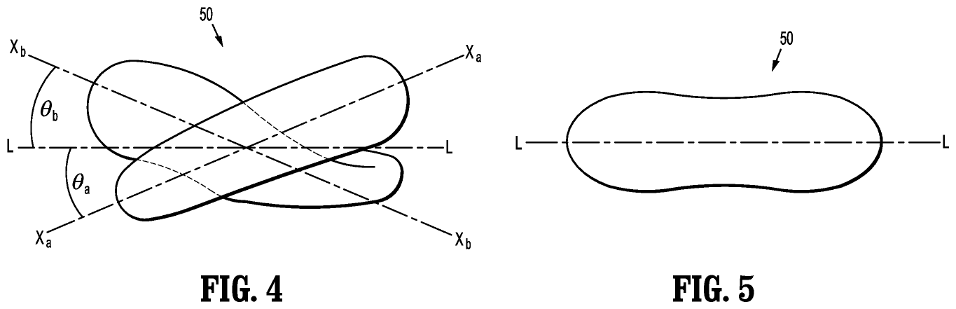

Further still, with additional reference to FIG. 4, as the bases 10a, 10b of the surgical fasteners 100a, 100b deform such that the distal tips 24a, 24b of the legs 20a, 20b of the surgical fasteners 100a, 100b reside proximate the second ends 14a, 14b of the bases 10a, 10b of the surgical fasteners 100a, 100b, the legs 20a, 20b of the surgical fasteners 100a, 100b cross over one another such that, when viewed from above (FIG. 4), the legs 20a, 20b of the surgical fasteners 100a, 100b define an X-shaped pattern. The mid points 28a, 28b of the legs overlap each other and define an orthogonal angle with respect to the junction 16 between the first ends 12a, 12b of the bases 10a, 10b of the surgical fasteners 100a, 100b. The junction 16 between the first ends 12a, 12b of the bases 10a, 10b of the surgical fasteners 100a, 100b lies along axis Y-Y (FIG. 3). The legs 20a, 20b of the surgical fasteners 100a, 100b are angled with respect to the longitudinal axis L-L of their bases 10a, 10b such that the longitudinal axis X-X of each leg 20a, 20b defines an acute angle Θ with respect to the longitudinal axis L-L of the bases 10a, 10b.

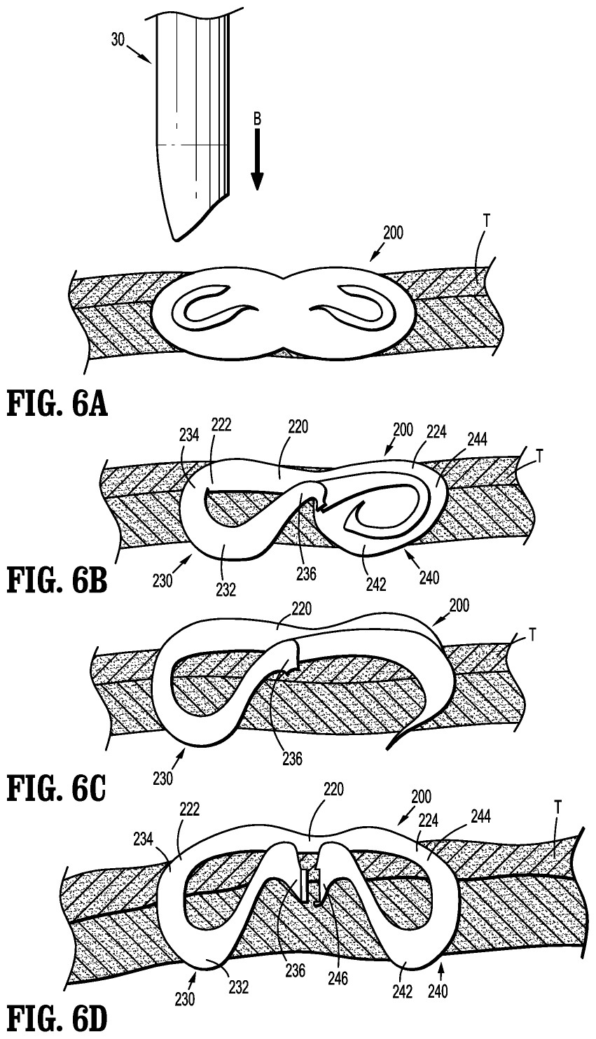

With reference now to FIGS. 6A-6D, another aspect of the present disclosure is illustrated. In particular, a surgical fastener 200 is shown with the actuator rod 30. The surgical fastener 200 is formed from a biocompatible material and, similar to surgical fasteners 100a, 100b, may be formed from a material having shape memory properties such as a shape memory metal (e.g., a nickel-titanium alloy or a copper-aluminum-nickel alloy) or a shape memory polymer. The surgical fastener 200 has a base 220 with first and second legs 230, 240 extending from opposed first and second ends 222, 224 of the base 220. The first and second legs 230, 240 have an arcuate configuration defined by first and arcuate second arms 232, 242. The first and second arcuate arms 232, 242 have respective first and second proximal ends 234, 244 that are resiliently attached to the opposed first and second ends 222, 224 of the base 220. The arcuate first and second arms 232, 242 include respective first and second distal ends 236, 246 that are spaced from the first and second proximal ends 234, 244 of the first and second arcuate arms 232, 242. The first and second arcuate arms 232, 242 are flexible such that they are transitionable between an undeployed configuration as seen in FIG. 6A and a deployed configuration as shown in FIG. 6D. FIGS. 6B and 6C depict intermediate configurations between the undeployed and deployed configurations.

Transitioning from the undeployed configuration shown in FIG. 6A to the deployed configuration shown in FIG. 6D involves the following steps. Initially, as seen in FIG. 6A, the surgical fastener 200 is positioned adjacent body tissue T. The surgical fastener 200 is in the undeployed configuration and the first leg 230 crosses over the second leg 240 such that the first and second legs 230, 240 overlap near a midpoint of the base 220. The actuator rod 30 is translated relative to the surgical fastener 200 in the direction of arrow “B” such that a distal portion of the actuator rod 30 slidably engages the first arm 232 of the first leg 230. As the first arm 232 is resiliently attached to first end 232 of the base 220, the engagement of the actuator rod 30 and the first leg 230 moves the first leg 230 relative to the base 220 towards an intermediate configuration as shown in FIG. 6B. As the first leg 230 transitions towards the deployed configuration, the first distal end 236 engages the second distal end 246 and urges the second leg 240 towards the deployed configuration as the first leg 230 is transitioning towards the deployed configuration. The actuator rod 30 is translated proximally away from the surgical fastener 200 and the first leg 230 is fully extended (FIG. 6C) while the second leg 240 continues to transition from the undeployed configuration to the deployed configuration. In the deployed configuration, each of the first and second legs 230, 240 defines an acute angle with respect to the base 220 of the surgical fastener. As depicted in FIG. 6D, the first and second legs 230, 240 are fully deployed such that the surgical fastener 200 has a generally B-shape with a notch in the middle between the first and second legs. The notch provides more traction and grip between the adjacent layers of tissue. In the deployed configuration, the surgical fastener 200 joins the adjacent layers of tissue T.

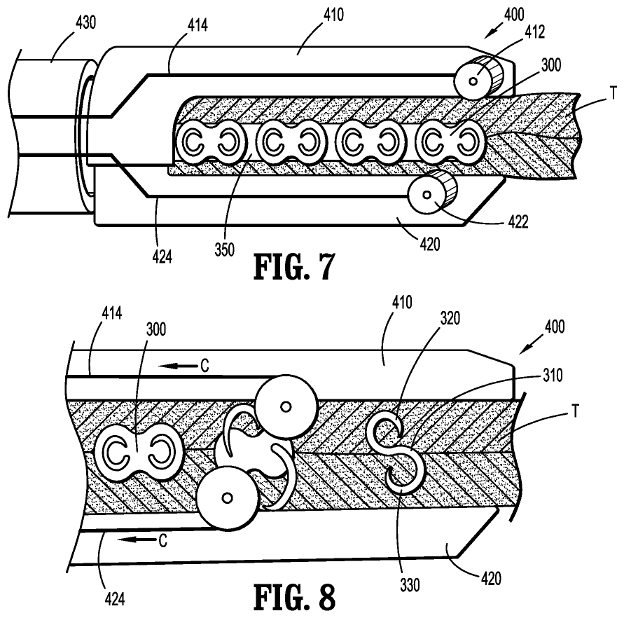

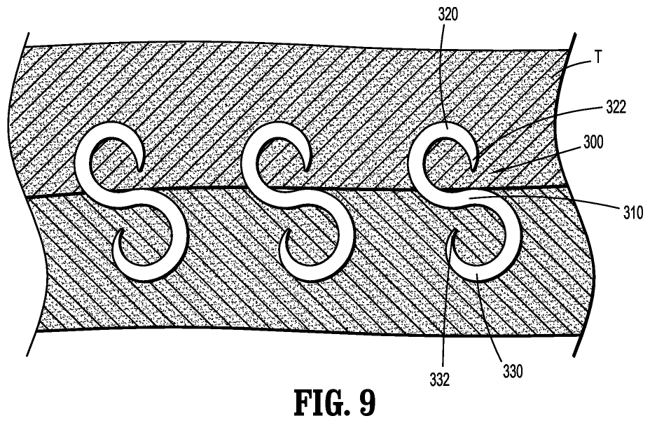

Turning now to FIGS. 7-9, another aspect of the present disclosure is illustrated. Surgical fasteners 300 are releasably supported on a ribbon 350 of a biocompatible material. Each surgical fastener 300 is formed from a biocompatible material and may be formed from a material having shape memory properties such as a shape memory metal (e.g., a nickel-titanium alloy or a copper-aluminum-nickel alloy) or a shape memory polymer. The ribbon 350 supporting the surgical fasteners 300 is positioned against layers of body tissue T as shown in FIG. 7. Each surgical fastener 300 includes a central portion 310 with first and second legs 320, 330 extending therefrom. The first and second legs 320, 330 have distal tips 322, 332. An end effector 400 includes opposed jaws 410, 420 that are in an approximated arrangement such that the adjacent layers of body tissue T are captured and retained therebetween. The jaws 410, 420 are repositionable between the approximated arrangement (FIG. 7) and an unapproximated arrangement (not shown) as known in the art. Jaws 410, 420 include respective rollers 412, 422 that are movable from a first or distal location in the jaws 410, 420 (FIG. 7) proximally towards a shaft 430 that supports the end effector 400 as indicated by arrow C. As shown, the rollers 412, 422 are connected to cables 414, 424. When the cables 414, 424 are translated proximally, they cause corresponding proximal movement of the rollers 412, 422. It is contemplated that the rollers 412, 422 may be spaced from the ribbon 350 and move towards each other and the ribbon 350 during an actuation sequence. The rollers 412, 422 are longitudinally staggered, but move proximally in unison. As the rollers 412, 422 are translated proximally, the rollers 412, 422 engage the legs 320, 330 respectively of the surgical fasteners 300. Engagement between the rollers 412, 422 and the legs 320, 330 of the surgical fasteners 300 moves the distal tips 322, 332 away from the central portion 310 of the surgical fastener 300 and through body tissue T. This transitions the surgical fasteners 300 from the undeployed configuration where the surgical fasteners 300 are stored on the ribbon 350 (FIG. 7) to the deployed configuration where the legs 320, 330 are spaced from the central portion 310 and the surgical fastener 300 has a generally S-shaped configuration (FIG. 9).

Persons skilled in the art will understand that the devices and methods specifically described herein and illustrated in the accompanying drawings are non-limiting. It is envisioned that the elements and features may be combined with the elements and features of another without departing from the scope of the disclosure. As well, one skilled in the art will appreciate further features and advantages of the disclosure.

What is claimed is:

1. A surgical fastener comprising:
a base having opposed first and second ends; and
a leg extending from the first end of the base, the leg including a distal tip configured to penetrate body tissue, the distal tip configured to extend towards the second end of the base, a length of the leg defining an acute angle with respect to a longitudinal axis of the base.

2. The surgical fastener according to claim 1, wherein the leg is resiliently coupled to the base.

3. The surgical fastener according to claim 1, wherein slidable engagement between the base and an actuator rod deflects the distal tip away from the second end of the base.

4. The surgical fastener according to claim 3, wherein the base is formed of a deformable material such that slidable engagement between the base and the actuator rod deforms the base thereby deflecting the distal tip of the leg.

5. The surgical fastener according to claim 2, wherein the leg of the first surgical fastener is configured to couple with a second surgical fastener having a base with opposed first and second ends and a leg resiliently coupled to the second end of the base.

6. The surgical fastener according to claim 5, wherein the distal tip of the leg contacts the second end of the base of the second surgical fastener.

7. The surgical fastener according to claim 1, wherein the surgical fastener is formed from a shape memory material.

8. A surgical fastening assembly comprising:
a first surgical fastener having a base and a leg, the base formed from a deformable material and having opposed first and second ends, the leg having a distal tip and a proximal end resiliently coupled to the second end of the base; and
a second surgical fastener having a base and a leg, the base formed from a deformable material and having opposed first and second ends, the leg having a distal tip and a proximal end resiliently coupled to the second end of the base,
wherein slidable engagement between the base of the first surgical fastener and an actuator rod deforms the base of the first surgical fastener thereby deflecting the distal tip of the leg of the first surgical fastener and slidable engagement between the base of the second surgical fastener and the actuator rod deforms the base of the second surgical fastener thereby deflecting the distal tip of the leg of the second surgical fastener.

9. The surgical fastening assembly according to claim 8, wherein slidable engagement between the actuator rod and the base of the second surgical fastener allows the distal tip of the leg of the first surgical fastener to transition to a rest position such that the distal tip of the leg of the first surgical fastener is proximate the second end of the base of the second surgical fastener.

10. The surgical fastening assembly according to claim 8, wherein the legs of the first and second surgical fasteners define acute angles with respect to a longitudinal axis of the first and second surgical fasteners.

11. The surgical fastening assembly according to claim 8, wherein the distal tips of the legs are configured to penetrate body tissue.

12. The surgical fastening assembly according to claim 10, wherein a mid point of the legs is orthogonal to junction between the first ends of the bases of the first and second surgical fasteners thereby defining a symmetrical surgical fastening assembly.

13. The surgical fastening assembly according to claim 12, wherein the legs cross one another and define an X-shape.

14. The surgical fastening assembly according to claim 9, wherein the first ends of the first and second surgical fasteners are in an abutting relationship.

15. The surgical fastening assembly according to claim 8, wherein the first and second surgical fasteners are formed from a shape memory material.

16. A surgical system for deploying a surgical fastener comprising:
an end effector having first and second jaws;
a ribbon supporting a surgical fastener thereon, the surgical fastener having a central portion with opposed first and second legs; and
first and second rollers disposed in the first and second jaws respectively, the first and second rollers attached to respective first and second cables, the first roller configured to engage the first leg of the surgical fastener and the second roller configured to engage the second leg of the surgical fastener such that proximal movement of the first and second rollers transitions the surgical fastener from an undeployed configuration to a deployed configuration.

17. The surgical system according to claim 16, wherein the surgical fastener is capable of joining adjacent layers of tissue in the deployed configuration.

18. The surgical system according to claim 17, wherein the surgical fastener has an S-shaped configuration in the deployed configuration.

19. The surgical system according to claim 16, wherein the surgical fastener is formed from a shape memory material.

20. The surgical system according to claim 16, wherein transitioning the surgical fastener from the undeployed configuration to the deployed configuration separates the surgical fastener from the ribbon.
